(19) United States
(12) Patent Application Publication
(10) Pub. No.: US 2016/0198934 A1
(43) Pub. Date: Jul. 14, 2016

(54) RESECTOSCOPE WITH LASER FIBERS

(71) Applicant: OLYMPUS WINTER & IBE GMBH, Hamburg (DE)

(72) Inventor: Nils KAPFERMANN, Hamburg (DE)

(73) Assignee: OLYMPUS WINTER & IBE GMBH, Hamburg (DE)

(21) Appl. No.: 14/914,115

(22) PCT Filed: Oct. 29, 2014
(86) PCT No.: PCT/EP2014/002905

§ 371 Date: Feb. 24, 2016

(30) Foreign Application Priority Data
Nov. 14, 2013 (DE) 10 2013 018 972.1

(51) Int. Cl. A61B 1/00; A61B 18/24; A61B 1/307

(52) U.S. Cl. CPC A61B 1/0014; A61B 1/00087; A61B 1/307; A61B 18/24; A61B 2018/00547

(57) ABSTRACT

A resectoscope having a laser fiber, which is mounted to be longitudinally displaceable and emits light at its distal end as well as having a carriage, which is mounted so that it is longitudinally displaceable on the resectoscope and has an elongated bore, which is stationary on the carriage in at least some regions and is designed to receive the laser fiber, and having a clamping device designed for connecting the laser fiber to the carriage by clamping, wherein the clamping device has a clamping body, which is mounted so that it is movable on the carriage across the bore and in which a region of the bore that is moved with the clamping body is arranged.

The invention relates to a resectoscope of the type defined in the preamble of Claim 1.

Resectoscopes are surgical instruments used for ablation of tissue in the body. They are designed with an elongated shaft according to the endoscopic principle and allow observation of the surgical field with a lens system and the respective illumination. Resectoscopes have a carriage that is longitudinally displaceable by hand and moves a resection instrument, such as, for example, a high-frequency loop electrode or a laser fiber emitting at the distal end. Resectoscopes are used in urology, for example, to reduce the size of the prostate.

A generic resectoscope is described in DE 196 18 399 A1, where the laser fiber is secured in the receiving bore of the carriage with a thumbwheel clamped on the carriage. This ensures accurate control of the laser fiber by the carriage.

One disadvantage of this known construction is the point attack of the clamping head on the laser fiber, which is very sensitive mechanically. This results in the possibility of unreliable clamping or damage to the laser fiber.

Resectoscopes with a laser fiber mounted so that it is longitudinally displaceable are known from the documents DE 198 26 311 and DE 20 2011 051 869 U1. In addition, the documents WO 2008/133707 A1 and DE 10 2004 007 120 B3 describe possibilities for clamping laser fibers on medical instruments.

The object of the present invention is to improve the clamping device in a generic resectoscope.

This object is achieved by the features of the characterizing part of claim 1.

According to the invention, a clamping body is supported on the carriage with a region of the receiving bore moving with the carriage. However, other regions of the receiving bore are arranged in a stationary position in the carriage. The laser fiber thus passes through at least one region of the receiving bore that is stationary on the carriage and a region of the receiving bore that is movable with the clamping body.

The regions of the receiving bore are displaced relative to one another by transverse movement of the clamping body, which thus results in the laser fiber becoming stuck. The laser fiber is then acted upon by the walls of the receiving bore over a large area, so that, with secure clamping, this results only in minor loads for the sensitive laser fiber.

The kinematics according to claim 2, in which the moving region of the receiving bore is moved, so that it executes an eccentric rotation with respect to the stationary region, are advantageous. This results in a very accurate controllability of the clamping forces, in particular when the eccentricity conforms to the conditions of claim 3.

It would be sufficient if only one stationary region of the receiving bore were arranged distally or proximally from the clamping body. However, according to claim 4, stationary receiving bores are advantageously provided on both sides, leading to a particularly secure symmetrical application of the clamping forces.

The embodiment of the invention according to claims 3 and 4 offers yet another advantage. It has kinematic and structural similarities with the German Patent Application DE 10 2012 023 275 A1, which was published subsequently and has a design for fastening an electrode on the carriage. The two designs can be combined with one another, wherein only the clamping body need be exchanged to switch a resectoscope from operation using a laser fiber to operation using an electrode.

The drawings show the invention schematically and as an example, in which:

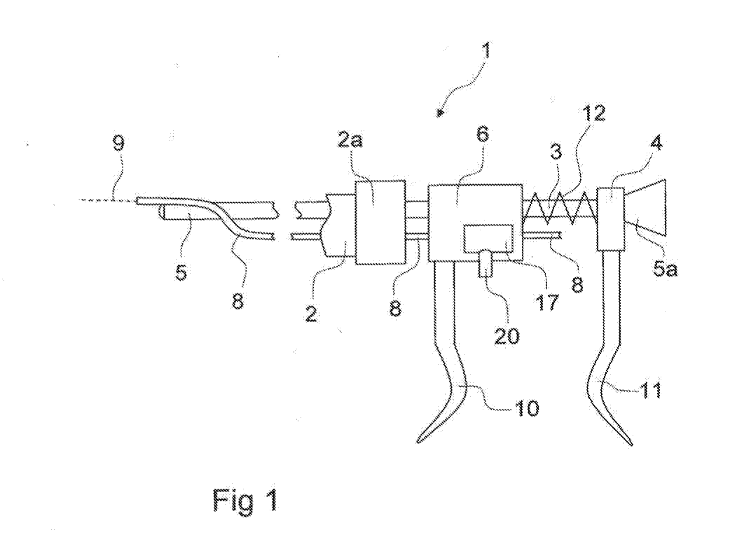

FIG. 1 shows a side view of an inventive resectoscope with a carriage,

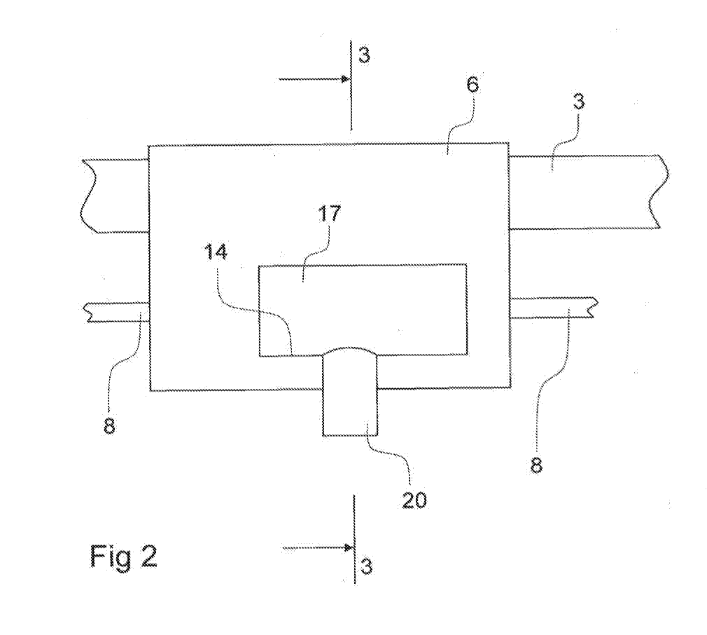

FIG. 2 shows an enlarged detail view of the carriage from FIG. 1,

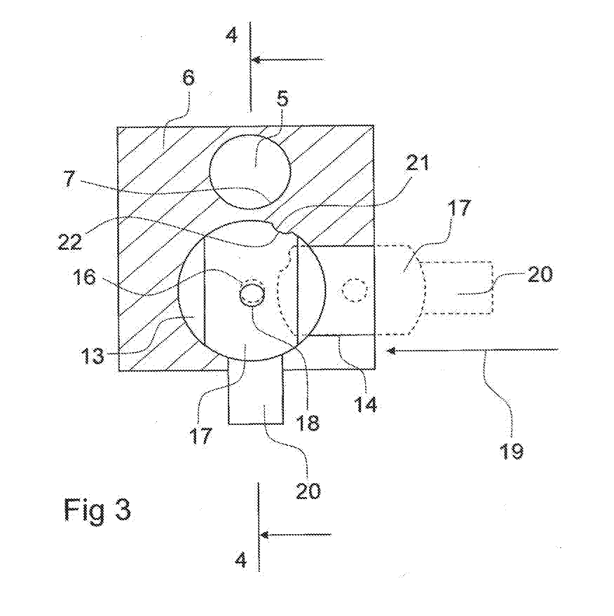

FIG. 3 shows a section according to line 3-3 in FIG. 2,

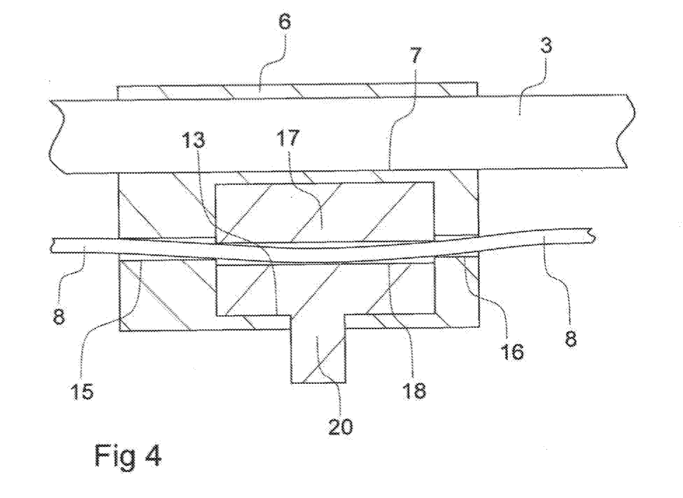

FIG. 4 shows a section according to line 4-4 in FIG. 3,

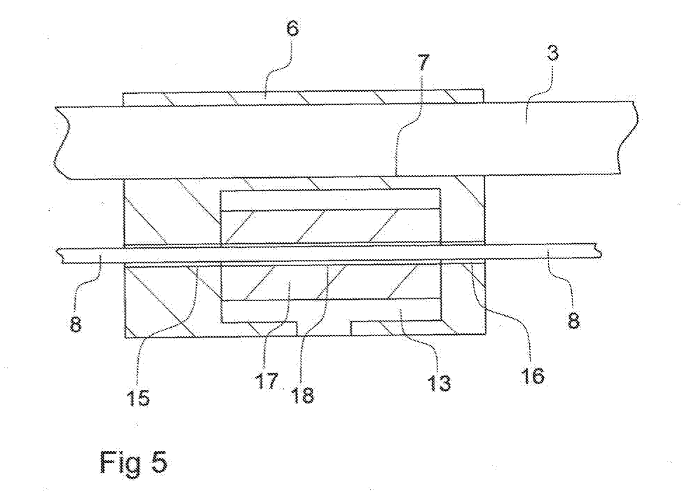

FIG. 5 shows a section according to FIG. 4, but with the clamping body rotated, and

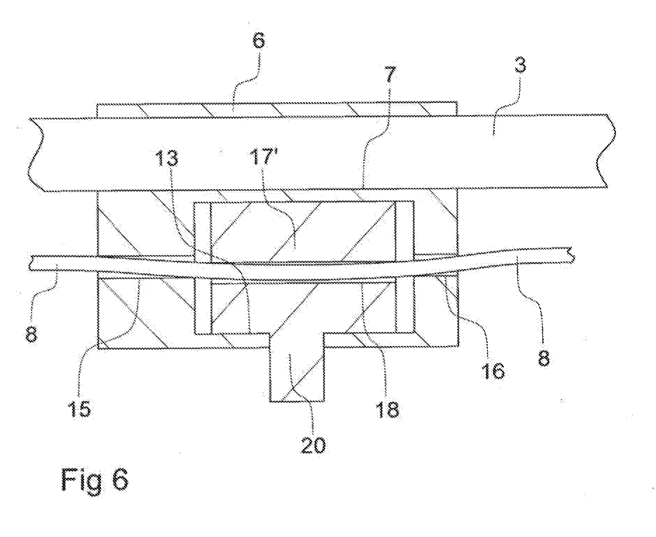

FIG. 6 shows a diagram corresponding to FIG. 4 in one embodiment variant.

FIG. 1 shows a resectoscope 1 with a shaft tube 2 fastened on a main body 2a. The main body is connected to a guide plate 4 by an optical guide tube 3.

An elongated lens 5 with an eyepiece 5a arranged on the proximal end passes through the guide tube 3 and the shaft tube 2 and is arranged in such a way that the surgical field in front of the distal end of the shaft tube 2 can be observed. A lighting device for illuminating the surgical field is not shown for the sake of simplicity of the drawing.

A carriage 6 is mounted so that it is displaceably guided on the guide tube 3 with a guide bore 7 between the main body 2a and the guide plate 4. As shown in FIGS. 1 and 2, a laser fiber 8 passes through it, passing through the slight bend in the shaft tube 2, as shown in FIG. 1, up to its distal end, so that it can emit a laser beam 9.

As FIG. 1 shows, the carriage 6 has a finger grip piece 10, and the guide plate 4 has a finger grip piece 11. By operating these two finger grip piece pieces 10 and 11 with the fingers of one hand, the carriage 6 can be displaced against the force of a spring 12, which supports the carriage 6 in the direction of displacement with respect to the guide plate 4.

The laser fiber 8 is to be moved in the longitudinal direction of the endoscope 1, i.e., in the direction of the lens 5 and/or of the shaft tube 2, by the movement of the carriage 6. Therefore, a mechanical fastening between the laser fiber 8 and the carriage 6 is required.

FIGS. 2 to 4 show the fastening of the laser fiber 8 on the carriage 6 in sectional diagrams.

A cylindrical cavity 13, whose axis runs in the longitudinal direction, is thus parallel to the guide bore 7 in the carriage 6. The cavity 13 is accessible through a slot 14 at the side.

As shown by the figures, the laser fiber 8 passes through the cylindrical cavity 13 and two bores 15 and 16 in the carriage 6, which are arranged distally and/or proximally from the cavity 13, so that they are arranged to be flush with one another and in the longitudinal direction, i.e., parallel to the guide bore 7.

In the embodiment shown here, the axis of the cylindrical cavity 13 is arranged to be concentric with the boreholes 15 and 16, of which the bore 16 is shown with dotted lines in FIG. 3.

The damping body 17 has a bore 18 in which the laser fiber 8 runs, passing through the clamping body 17 in parallel to the bores 15 and 16 but eccentrically in relation to them.

The clamping body 17 is mounted in the cavity 13, so that it can rotate about the axis, as shown in FIG. 3. This bearing support is accomplished with its circumferential surfaces, but as shown in FIG. 3, the clamping body 17 is flattened on two parallel sides, wherein these parallel sides are situated at a distance corresponding to the width of the slot 14. FIG. 3 shows the clamping body 17 in a diagram with dotted lines in a position rotated by 90 degrees, in which it fits through the slot 14. When the clamping body is inserted into the region of the cavity 13 in the direction of the arrow 19, it can then be rotated about its axis in the cavity 13 into the position represented by solid lines in FIG. 3. For the rotation, an actuating lever 20 that protrudes at the side may be gripped from the outside. The damping body 17 has a groove 21 in its otherwise cylindrical outside surface. A cam 22 protruding inward is situated in the inside surface of the otherwise cylindrical cavity 13. In the position of the clamping body 17, which is rotated by 90 degrees with respect to the inserted position (dotted lines), the groove 21 and the cam 22 enter the engaged position illustrated in FIG. 3. In this rotational position, the clamping body is thus held by catch engagement.

FIG. 3 shows that the region 18 of the bore 15, 18, 16 through which the carriage 6 passes, said region moving with the clamping body 17, is designed to be eccentric with the stationary regions 15, 16 of the bore. In the rotational position of the clamping body 17 shown with solid lines in FIG. 3 with the lever 20 pointing downward, the bore 18 is displaced eccentrically with respect to the bores 15 and 16. FIG. 4 illustrates this rotational position.

If the clamping body 17 is rotated by 90 degrees into the rotational position shown with dotted lines, then all three bores 15, 16 and 18 are aligned as shown in FIG. 5. It can be seen that in the position shown in FIG. 5, the laser fiber 8 can be displaced freely by means of the carriage 6, i.e., in particular being inserted during assembly and brought into its target position.

If the clamping body 17 then is rotated out of the rotational position in FIG. 5 and into the rotational position in FIG. 4, it can be seen that through the eccentric displacement of the boreholes, the laser fiber is clamped. It should be noted that the eccentricity is less than the diameter of the boreholes 15, 16 and 18 because otherwise there would not be a remaining free passage channel. The eccentricity must of course also be adapted to the diameter of the laser fiber, i.e., approximately as shown in FIG. 4. Then a slight, non-damaging clamping is achieved in the position shown in FIG. 4.

In a diagram according to FIG. 4, FIG. 6 shows an embodiment variant in which the clamping body 17′ is somewhat shorter than the cavity 13 in the direction of the laser fiber 8. There is thus a distance between the ends of the moving bore 18 and the internal ends of the bores 15 and 16 that are stationary with respect to the carriage 6. As can be seen from FIG. 6, this yields a reduced shearing effect between these bore ends and thus a reduced risk of damage.

### LIST OF REFERENCE NUMERALS

1 resectoscope
2 shaft tube
2a main body
3 guide tube
4 guide plate
5 lens
5a eyepiece
6 carriage
7 guide bore
8 laser fiber
9 laser beam
10 finger grip piece
11 finger grip piece
12 spring
13 cavity
14 slot
15 bore
16 stationary bore
17 stationary clamping body
18 moving bore
19 arrow
20 lever
21 groove
22 cam

1. A resectoscope having a laser fiber, which is mounted so that it is longitudinally displaceable and emits from its distal end, and having a carriage, which is mounted to be longitudinally displaceable on the resectoscope and has an elongated bore designed to receive the laser fiber and is stationary in at least some regions on the carriage and also has a clamping device designed for connecting the laser fiber to the carriage by clamping, wherein the clamping device has a clamping body, which is mounted on the carriage, so that it is movable across the bore, a region of the bore that is moved with the clamping body being arranged therein.

2. The resectoscope according to claim 1, wherein the clamping body is mounted in the carriage so that it can be rotated about an axis parallel to the stationary region of the bore, wherein the moving region of the bore is arranged in the clamping body so that it is eccentric with the axis.

3. The resectoscope according to claim 2, wherein the axis is arranged to be coaxial with the stationary region of the bore, and the eccentricity is less than the diameter of the bore.

4. The resectoscope according to claim 2, wherein stationary regions of the bore are arranged distally and proximally from the clamping body in the carriage.

5. The resectoscope according to claim 3, wherein stationary regions of the bore are arranged distally and proximally from the clamping body in the carriage.
